(19) United States
(12) Patent Application Publication
(10) Pub. No.: US 2008/0149472 A1
(43) Pub. Date: Jun. 26, 2008

(54) Photochlorination and Fluorination Process for Preparation of Fluorine-Containing Hydrocarbons

(76) Inventors: Velliyur Nott Mallikarjuna Rao, Wilmington, DE (US); Allen Capron Sievert, Elkton, MD (US)

Correspondence Address: David E. Heiser;E.I. Du Pont De Nemours and Company, Legal Patent Records Center, 4417 Lancester Pike, Wilmington, DE, 19805, US

(21) Appl. No.: 11/792,643

(22) PCT Filed: Dec. 19, 2005
(86) PCT No.: PCT/US05/46267

§ 371 Date: Jun. 7, 2007

Related U.S. Application Data
(60) Provisional application No. 60/638,289, filed on Dec. 22, 2004.

(51) Int. Cl. C07C 17/00

(52) U.S. Cl. 204/157.95

(57) ABSTRACT

A process is disclosed for increasing the fluorine content of at least one compound selected from halohydrocarbons and hydrocarbons. The process involves (a) directing light from a light source through the wall of a reactor to interact with reactants comprising chlorine and said at least one compound in said reactor, thereby producing a halogenated hydrocarbon having increased chlorine content by photochlorination, and (b) reacting said halogenated hydrocarbon produced by the photochlorination in (a) with HF; and is characterized by the light directed through the reactor wall being directed through a poly(perhaloolefin) polymer.

### FIELD OF THE INVENTION

This invention relates to the field of fluorinating chlorine-containing compounds, and particularly to materials suitable for use in producing the chlorine-containing compounds used in fluorination by a photochlorination process.

### BACKGROUND OF THE INVENTION

Photochemical reactions use light as a source of energy to promote chemical processes. Ultraviolet (UV) and visible light are widely used in chemical synthesis both in laboratories and in commercial manufacturing. Well known photochemical reactions include photodimerization, photopolymerization, photohalogenation, photoisomerization and photodegradation. For example, cyclobutanetetracarboxylic dianhydride can be synthesized by photodimerization of maleic anhydride in a glass reactor using a mercury UV lamp (P. Boule et al., Tetrahedron Letters, Volume 11, pages 865 to 868, (1976)). Most of the vitamin D production in the United States is based on UV photolysis in a quartz vessel using light between 275 and 300 nm.

In photochlorination, chlorine (Cl₂) reacts with a saturated or unsaturated starting material, in the presence of a ultraviolet light source. This process is widely used to form carbon-chlorine bonds under mild conditions (e.g., room temperature) compared to the elevated temperatures normally required for thermal chlorination (R. Roberts et al., Applications of Photochemistry, TECHNOMIC Publishing Co., Inc. 1984.). For example, E. Tschuikow-Roux, et al. (J. Phys. Chem., Volume 88, pages 1408 to 1414 (1984)) report photochlorination of chloroethane and Walling et al. (J. Amer. Chem. Soc., Volume 79, pages 4181 to 4187 (1957)) report photochlorination of certain substituted toluenes. U.S. Pat. No. 5,190,626 describes the use of photochlorination in removing unsaturated compounds such as vinylidine chloride from CCl₂FCH₃ product. Chlorine-containing compounds such as CCl₂FCH₃ may be readily converted to fluorine-containing compounds (e.g., CF₃CH₃) by fluorination using hydrogen fluoride (HF).

Typically in photochlorinations, light from a suitable source (e.g., an incandescent bulb or a UV lamp) is directed through a reactor wall to interact with the reactants therein. The portion of the reactor wall through which the light passes must have a suitable transmittance to allow light of a wavelength required for the photochlorination to enter the reactor. Typically, quartz or borosilicate glass like Pyrex™ glass have been employed as transparent materials. Quartz is expensive, but has a low cut-off wavelength at about 160 nm; Pyrex™ glass is less expensive, but has a relatively high cut-off wavelength at about 275 nm. Due to their reactivity, quartz and Pyrex are not appropriate materials of construction for chemical reactions involving base or HF. There is a need for additional materials which can be used for this purpose in photochemical reactions (e.g., photochlorinations).

### SUMMARY OF THE INVENTION

This invention provides a process for increasing the fluorine content of at least one compound selected from halohydrocarbons and hydrocarbons. The process comprises (a) directing light from a light source through the wall of a reactor to interact with reactants comprising chlorine and said at least one compound in said reactor, thereby producing a halogenated hydrocarbon having increased chlorine content by photochlorination, and (b) reacting said halogenated hydrocarbon produced by the photochlorination in (a) with HF. In accordance with this invention, the process is characterized by the light directed through the reactor wall being directed through a poly(perhaloolefin) polymer.

### DETAILED DESCRIPTION

In accordance with this invention poly(perhaloolefin) polymers are used as photochlorination reactor materials through which light is able to pass for the purpose of interacting with the reactants, thereby promoting the photochlorination reaction. Preferred poly(perhaloolefin) polymers include perfluorinated polymers. Of note are embodiments where the poly(perhaloolefin) polymer is PTFE (i.e., poly(tetrafluoroethylene)). Also of note are embodiments where the poly(perhaloolefin) polymer is FEP (i.e., a copolymer of tetrafluoroethylene with hexafluoropropylene).

Perfluoropolymers have excellent chemical resistance, low surface energy, low flammability, low moisture adsorption, excellent weatherability and high continuous use temperature. In addition, they are among the purest polymer materials and are widely used in the semiconductor industry. They are also excellent for UV-vis transmission. For instance, a film of PFA copolymer (copolymers of tetrafluoroethylene and perfluoroalkyl vinyl ether) having a thickness of 0.025 mm has transmission of 91-96% for visible light between 400 to 700 nm and transmission of 77-91% for UV light between 250 to 400 nm. Transmission of visible light through FEP is similar to PFA and UV light transmission of FEP is slightly better than PFA.

A suitable photochlorination apparatus includes a reactor in which light having a suitable wavelength (e.g., from about 250 nm to about 400 nm) can irradiate the reaction components for a time sufficient to convert at least a portion of the starting materials to one or more compounds having a higher chlorine content. The reactor may be, for example, a tubular reactor fabricated from poly(perhaloolefin) polymer (e.g., either a coil or extended tube), or tank fabricated from poly(perhaloolefin) polymer, or a tube or tank fabricated from an opaque material which has a window fabricated from poly(perhaloolefin) polymer. Typically, the thickness of the poly(perhaloolefin) polymer is sufficient to permit transmittance of the light of sufficient intensity to promote the reaction (e.g., 0.02 mm to 1 mm). Where additional structural reinforcement is desired while maintaining the chemical resistance offered by the poly(perhaloolefin) polymer, a layer of reinforcing material fabricated from a highly transmitting material (e.g., quartz) or a mesh of transmitting or opaque material may be used outside of the poly(perhaloolefin) polymer layer.

The apparatus also includes a light source. The light source may be any one of a number of arc or filament lamps known in the art. The light source is situated such that light having the desired wavelength may introduced into the reaction zone (e.g., a reactor wall or window fabricated from a poly(perhaloolefin) polymer and suitably transparent to light having a wavelength of from about 250 nm to about 400 nm).

Ordinarily the apparatus also includes a chlorine (Cl₂) source and a source of the material to be chlorinated. The chlorine source may be, for example, a cylinder containing chlorine gas or liquid, or equipment that produces chlorine (e.g., an electrochemical cell) that is connected to the reactor. The source of the material to be chlorinated may be, for example, a cylinder or pump fed from a tank containing the material, or a chemical process that produces the material to be chlorinated.

Increasing Chlorine Content

In step (a) of the process of this invention the chlorine content of a halogenated hydrocarbon compound or a hydrocarbon compound is increased by reacting said compound with chlorine (Cl₂) in the presence of light.

Halogenated hydrocarbon compounds suitable as starting materials for the chlorination process of this invention may be saturated or unsaturated. Saturated halogenated hydrocarbon compounds suitable for the chlorination processes of this invention include those of the general formula CₙHₐBr_{b}Cl_{c}F_{d}, wherein n is an integer from 1 to 4, a is an integer from 1 to 9, b is an integer from 0 to 4, c is an integer from 0 to 9, d is an integer from 0 to 9, the sum of b, c and d is at least 1 and the sum of a, b, c, and d is equal to 2n+2. Saturated hydrocarbon compounds suitable for chlorination are those which have the formula C_{q}Hᵣ where q is an integer from 1 to 4 and r is 2q+2. Unsaturated halogenated hydrocarbon compounds suitable for the chlorination processes of this invention include those of the general formula CₚHₑBr_{f}Cl_{g}Fₕ, wherein p is an integer from 2 to 4, e is an integer from 0 to 7, f is an integer from 0 to 2, g is an integer from 0 to 8, h is an integer from 0 to 8, the sum of f, g and h is at least 1 and the sum of e, f, g, and h is equal to 2p. Unsaturated hydrocarbon compounds suitable for chlorination are those which have the formula CᵢHⱼ where i is an integer from 2 to 4 and j is 2i. The chlorine content of saturated compounds of the formula CₙHₐBr_{b}Cl_{c}F_{d} and C_{q}Hᵣ and/or unsaturated compounds of the formula CₚHₑBr_{f}Cl_{g}Fₕ and CᵢHⱼ may be increased by reacting said compounds with Cl₂ in the vapor phase in the presence of light. Such a process is referred to herein as a photochlorination reaction.

The photochlorination of the present invention may be carried out in either the liquid or the vapor phase. For vapor phase photochlorination, initial contact of the starting materials with Cl₂ may be a continuous process in which one or more starting materials are vaporized (optionally in the presence of an inert carrier gas, such as nitrogen, argon, or helium) and contacted with chlorine vapor in a reaction zone. A suitable photochlorination reaction zone is one in which light having a wavelength of from about 250 nm to about 400 nm can irradiate the reaction components for a time sufficient to convert at least a portion of the starting materials to one or more compounds having a higher chlorine content. The source of light may be any one of a number of arc or filament lamps known in the art. Light having the desired wavelength may introduced into the reaction zone by a number of means. For example, the light may enter the reaction zone through a lamp well or window fabricated from a poly(perhaloolefin) polymer suitably transparent to light having a wavelength of from about 250 nm to about 400 nm. Likewise, the walls of the reaction zone may be fabricated from such a material so that at least a portion of the light used for the photochlorination can be transmitted through the walls.

Alternatively, the process of the invention may be carried out in the liquid phase by feeding Cl₂ to a reactor containing the starting materials. Suitable liquid phase reactors include vessels fabricated from a poly(perhaloolefin) polymer in which an external lamp is directed toward the reactor and metal, glass-lined metal or fluoropolymer-lined metal reactors having one or more wells or windows fabricated from a poly(perhaloolefin) polymer for introducing light having a suitable wavelength. Preferably the reactor is provided with a condenser or other means of keeping the starting materials in the liquid state while permitting the hydrogen chloride (HCl) released during the chlorination to escape the reactor.

In some embodiments it may be advantageous to conduct the photochlorination in the presence of a solvent capable dissolving one or more of the starting materials and/or chlorination products. Preferred solvents include those that do not have easily replaceable hydrogen substituents. Examples of solvents suitable for step (a) include carbon tetrachloride, 1,1-dichlorotetrafluoroethane, 1,2-dichlorotetrafluoroethane, 1,1,2-trichlorotrifluoroethane, benzene, chlorobenzene, dichlorobenzene, fluorobenzene, and difluorobenzene.

Suitable temperatures for the photochlorination of the starting materials of the formula are typically within the range of from about −20° C. to about 60° C. Preferred temperatures are typically within the range of from about 0° C. to about 40° C. In the liquid phase embodiment, it is convenient to control the reaction temperature so that starting material is primarily in the liquid phase; that is, at a temperature that is below the boiling point of the starting material(s) and product(s).

The pressure in a liquid phase process is not critical so long as the liquid phase is maintained. Unless controlled by means of a suitable pressure-regulating device, the pressure of the system increases as hydrogen chloride is formed by replacement of hydrogen substituents in the starting material by chlorine substituents. In a continuous or semi-batch process it is possible to set the pressure of the reactor in such a way that the HCl produced in the reaction is vented from the reactor (optionally through a packed column or condenser). Typical reactor pressures are from about 14.7 psig (101.3 kPa) to about 50 psig (344.6 kPa).

The amount of chlorine (Cl₂) fed to the reactor is based on whether the starting material(s) to be chlorinated is(are) saturated or unsaturated, and the number of hydrogens in CₙHₐBr_{b}Cl_{c}F_{d}, C_{q}Hᵣ, CₚHₑBr_{f}Cl_{g}Fₕ, and CᵢHⱼ that are to be replaced by chlorine. One mole of Cl₂ is required to saturate a carbon-carbon double bond and a mole of Cl₂ is required for every hydrogen to be replaced by chlorine. A slight excess of chlorine over the stoichiometric amount may be necessary for practical reasons, but large excesses of chlorine will result in complete chlorination of the products. The ratio of Cl₂ to halogenated carbon compound is typically from about 1:1 to about 10:1.

Specific examples of photochlorination reactions of saturated halogenated hydrocarbon compounds of the general formula CₙHₐBr_{b}Cl_{c}F_{d} and saturated hydrocarbon compounds of the general formula C_{q}Hᵣ which may be carried out in accordance with this invention include the conversion of C₂H₆ to a mixture containing CH₂ClCCl₃, the conversion of CH₂ClCF₃ to a mixture containing CHCl₂CF₃, the conversion of CCl₃CH₂CH₂Cl, CCl₃CH₂CHCl₂, CCl₃CHClCH₂Cl or CHCl₂CCl₂CH₂Cl to a mixture containing CCl₃CCl₂CCl₃, the conversion of CH₂FCF₃ to a mixture containing CHClFCF₃ and CCl₂FCF₃, the conversion of CH₃CHF₂ to CCl₃CClF₂, the conversion of CF₃CHFCHF₂ to a mixture containing CF₃CClFCHF₂ and CF₃CHFCClF₂, and the conversion of CF₃CH₂CHF₂ to CF₃CH₂CClF₂.

Specific examples of photochlorination reactions of unsaturated halogenated hydrocarbon compounds of the general formula CₚHₑBr_{f}Cl_{g}Fₕ and unsaturated hydrocarbon compounds of the general formula CᵢHⱼ which may be carried out in accordance with this invention include the conversion of C₂H₄ to a mixture containing CH₂ClCH₂Cl, the conversion of C₂Cl₄ to a mixture containing CCl₃CCl₃, the conversion of C₃H₆ a mixture containing CCl₃CCl₂CCl₃, and the conversion of CF₃CCl═CCl₂ to a mixture containing CF₃CCl₂CCl₃.

Of note is a photochlorination process for producing a mixture containing 2-chloro-1,1,1-trifluoroethane (i.e., CH₂ClCF₃ or HCFC-133a) by reaction of CH₃CF₃ with Cl₂ in the vapor phase in the presence of light in accordance with this invention. Also of note is a catalytic process for producing a mixture containing 1,2,2-trichloro-1,1,3,3,3-pentafluoropropane (i.e., CClF₂CCl₂CF₃ or CFC-215aa) or 1,2-dichloro-1,1,1,3,3,3-hexafluoropropane (i.e., CClF₂CClFCF₃ or CFC-216ba) by the chlorination of a corresponding hexahalopropene of the formula C₃Cl₆₋ₓFₓ, wherein x equals 5 or 6.

Contact times of from 0.1 to 60 seconds are typical; and contact times of from 1 to 30 seconds are often preferred.

Mixtures of saturated hydrocarbon compounds and saturated halogenated hydrocarbon compounds and mixtures of unsaturated hydrocarbon compounds and unsaturated halogenated hydrocarbon compounds as well as mixtures comprising both saturated and unsaturated compounds may be chlorinated in accordance with the present invention. Specific examples of mixtures of saturated and unsaturated hydrocarbons and halogenated hydrocarbons that may be used include a mixture of CCl₂═CCl₂ and CCl₂═CClCCl₃, a mixture of CHCl₂CCl₂CH₂Cl and CCl₃CHClCH₂Cl, a mixture of CHCl₂CH₂CCl₃ and CCl₃CHClCH₂Cl, a mixture of CHCl₂CHClCCl₃, CCl₃CH₂CCl₃, and CCl₃CCl₂CH₂Cl, a mixture of CHF₂CH₂CF₃ and CHCl═CHCF₃, and a mixture of CH₂═CH₂ and CH₂═CHCH₃.

Increasing the Fluorine Content

In step (b) of the process of this invention the halogenated hydrocarbon produced in step (a) is reacted with hydrogen fluoride. Fluorination reactions are well known in the art. They can be conducted both in either the vapor phase or liquid phase using a variety of fluorination catalysts. See for example, Milos Hudlicky, Chemistry of Organic Fluorine Compounds 2^{nd} (Revised Edition), pages 91 to 135 and references cited therein (Ellis Harwood-Prentice Hall Publishers, 1992). Of note are vapor phase fluorinations in the presence of a fluorination catalyst. Preferred fluorination catalysts include chromium catalysts (e.g., Cr₂O₃ by itself of with other metals such as magnesium halides or zinc halides on Cr₂O₃); chromium(III) halides supported on carbon; mixtures of chromium and magnesium (including elemental metals, metal oxides, metal halides, and/or other metal salts) optionally on graphite; and mixtures of chromium and cobalt (including elemental metals, metal oxides, metal halides, and/or other metal salts) optionally on graphite, alumina, or aluminum halides such as aluminum fluoride.

Fluorination catalysts comprising chromium are well known in the art (see e.g., U.S. Pat. No. 5,036,036). Chromium supported on alumina can be prepared as described in U.S. Pat. No. 3,541,834. Chromium supported on carbon can be prepared as described in U.S. Pat. No. 3,632,834. Fluorination catalysts comprising chromium and magnesium may be prepared as described in Canadian Patent No. 2,025,145. Other metals and magnesium optionally on graphite can be prepared in a similar manner to the latter patent.

Preferred chromium fluorination catalysts comprise trivalent chromium. Of note is Cr₂O₃ prepared by pyrolysis of (NH₄)₂Cr₂O₇, Cr₂O₃ having a surface area greater than about 200 m²/g, and Cr₂O₃ prepared by pyrolysis of (NH₄)₂Cr₂O₇ or having a surface area greater than about 200 m²/g some of which are commercially available.

Halogenated hydrocarbon compounds suitable for the fluorination of this invention include saturated compounds of the general formula CₘH_{w}BrₓCl_{y}F_{z}, wherein m is an integer from 1 to 4, w is an integer from 0 to 9, x is an integer from 0 to 4, y is an integer from 1 to 10, z is an integer from 0 to 9, and the sum of w, x, y, and z is equal to 2n+2.

Examples of saturated compounds of the formula CₘH_{w}BrₓCl_{y}F_{z} which may be reacted with HF in the presence of a catalyst include CH₂Cl₂, CHCl₃, CCl₄, C₂Cl₆, C₂BrCl₅, C₂Cl₅F, C₂Cl₄F₂, C₂Cl₃F₃, C₂Cl₂F₄, C₂ClF₅, C₂HCl₅, C₂HCl₄F, C₂HCl₃F₂, C₂HClF₄, C₂HBrF₄, C₂H₂Cl₄, C₂H₂Cl₃F, C₂H₂Cl₂F₂, C₂H₂ClF₃, C₂H₃Cl₃, C₂H₃Cl₂F, C₂H₃ClF₂, C₂H₄Cl₂, C₂H₄ClF, C₃Cl₆F₂, C₃Cl₅F₃, C₃Cl₄F₄, C₃Cl₃F₅, C₃HCl₇, C₃HCl₆F, C₃HCl₅F₂, C₃HCl₄F₃, C₃HCl₃F₄, C₃HCl₂F₅, C₃H₂Cl₆, C₃H₂BrCl₅, C₃H₂Cl₅F, C₃H₂Cl₄F₂, C₃H₂Cl₃F₃, C₃H₂Cl₂F₄, C₃H₂ClF₅, C₃H₃Cl₅, C₃H₃Cl₄F, C₃H₃Cl₃F₂, C₃H₃Cl₂F₃, C₃H₃ClF₄, C₃H₄Cl₄, C₄Cl₄Cl₄, C₄Cl₄Cl₆, C₄H₅Cl₅ and C₄H₅Cl₄F.

Of note is a process for producing 1,1,1,2,2-pentafluoroethane (i.e., CHF₂CF₃ or HFC-125) by the photochlorination of 1,1,1,2 tetrafluoroethane (i.e., CH₂FCF₃ or HFC-134a) to produce 2-chloro-1,1,1,2 tetrafluoroethane (i.e., CHClFCF₃ or HCFC-124); and the fluorination of the HCFC-124 to produce HFC-125. HFC-125 may also be produced by the photochlorination of 1,1,2,2 tetrafluoroethane (i.e., CHF₂CHF₂ or HFC-134) to produce 2-chloro-1,1,2,2 tetrafluoroethane (i.e., CClF₂CHF₂ or HCFC-124a); and fluorination of the HCFC-124a to produce HFC-125. Also of note is a process for producing 1,1,1,3,3,3-hexafluoropropane (CF₃CH₂CF₃ or HFC-236fa) by the photochlorination of 1,1,1,3,3-pentafluoropropane (CF₃CH₂CHF₂ or HFC-245fa) to produce 3-chloro-1,1,1,3,3-pentafluoropropane (i.e. CF₃CH₂CClF₂ or HCFC-235fa); and fluorination of the HCFC-235fa to produce HFC-236fa. Further discussion of producing HFC-236fa by fluorination is provided in U.S. Patent Application No. 60/638,277 which was filed Dec. 22, 2004, and is incorporated herein by reference.

In one embodiment of the invention, the photochlorination and further fluorination can be conducted in situ and the fluorinated product(s) recovered. In a second embodiment, the effluent from the photochlorination step may be fed to a second reactor for fluorination. The photochlorination product mixture can be fed to a fluorination reactor with or without prior separation of the products from the photochlorination reactor. Of note are processes where the photochlorination product mixture is directly fed to a fluorination reactor without prior separation of the products from the photochlorination reactor. In a third embodiment HF can be fed together with chlorine and the other photochlorination starting materials to the photochlorination reactor and the effluent from the photochlorination reactor can be directed to a fluorination zone optionally containing a fluorination catalyst; and additional HF, if desired, can be fed to the fluorination zone.

### EXAMPLES

### General Procedure for Chlorination and Product Analysis

Photochlorination was carried out using a 110 volt/275 watt sunlamp placed (unless otherwise specified) at a distance of 0.5 inches (1.3 cm) from the outside of the first turn of the inlet end of a coil of fluoropolymer tubing material through which the materials to be chlorinated were passed. Two fluoropolymer tubes were used in the examples below. One tube was fabricated from the PTFE (18 inches (45.7 cm) long× 1/16″ (16 mm) OD×0.038″ (0.97 mm) ID) which was coiled to a diameter of 2.5 inches (6.4 cm) and contained suitable feed and exit ports. The other tube was fabricated from FEP (18 inches (45.7 cm)×0.125″ (3.2 mm) OD×0.085″ (2.2 mm) ID) which was coiled to a diameter of 3 inches (7.6 cm) and contained suitable feed and exit ports. The organic feed material and chlorine were fed to the tubing using standard flow-measuring devices. The gas mixture inside was exposed to light generated by the sunlamp. The experiments were conducted at ambient temperature (about 23° C.) and under about atmospheric pressure. Organic feed material entering the tubing and the product after photochlorination were analyzed on-line using a GC/MS. The results are reported in mole %.

PTFE (poly(tetrafluoroethylene) is a linear homopolymer of tetrafluoroethylene (TFE). FEP is a copolymer of tetrafluoroethylene and hexafluoropropylene. CFC-114 is CClF₂CClF₂. CFC-114a is CF₃CCl₂F. HCFC-132b is CClF₂CH₂Cl. HCFC-142 is CHF₂CHCl₂. CFC-216ba is CF₃CClFCClF₂. HCFC-226ba is CF₃CClFCHF₂. HCFC-226ea is CF₃CHFCClF₂.

### Example 1

### Photochlorination of HFC-134a

Feed gases consisting of HFC-134a at a flow rate of 5.0 sccm (8.3(10)⁻⁸ m³/sec) and chlorine gas at a flow rate of 2.5 sccm (4.2(10)⁻⁷ m³/sec) were introduced into the PTFE tubing. After exposure to light for one hour, the product was analyzed and found to contain 68.1 mole % of HFC 134a, 24.2 mole % of HCFC-124, 7.0 mole % of CFC-114a and 0.7 mole % of other unidentified compounds. The molar yield of CFC-114a compared to the total amount of CFC-114a and HCFC-124 was 22.4%.

### Example 2

### Photochlorination of HFC-134a

Feed gases consisting of HFC-134a at a flow rate of 5.0 sccm (8.3(10)⁻⁸ m³/sec) and chlorine gas at a flow rate of 7.5 sccm (1.3(10)⁻⁷ m³/sec) were introduced into the PTFE tubing. After exposure to light for one hour, the product was analyzed and found to contain 61.4 mole % of HFC-134a, 27.5 mole % of HCFC-124, 10.7 mole % of CFC-114a and 0.4 mole % of other unidentified compounds. The molar yield of CFC-114a compared to the total amount of CFC-114a and HCFC-124 was 28.0%.

### Example 3

### Photochlorination of HFC-134a

Feed gases consisting of HFC-134a at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 53.0 mole % of HFC-134a, 30.0 mole % of HCFC-124, 16.2 mole % of CFC-114a and 0.8 mole % of other unidentified compounds. The molar yield of CFC-114a compared to the total amount of CFC-114a and HCFC-124 was 35.0%.

### Example 4

### Photochlorination of HFC-134a

In this experiment, the distance of the lamp from the coiled tube was 1.5 inches (3.8 cm). Feed gases consisting of HFC-134a at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 56.5 mole % of HFC-134a, 29.0 mole % of HCFC-124, 14.0 mole % of HCFC 114a and 0.5 mole % of other unidentified compounds. The molar yield of HCFC 114a compared to the total amount of HCFC 114a and HCFC 124 was 32.6%.

### Example 5

### Photochlorination of HFC 134a

In this experiment, the distance of the lamp from the coiled tube was 3.0 inches (7.6 cm). Feed gases consisting of HFC-134a at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 63.8 mole % of HFC-134a, 26.0 mole % of HCFC-124, 9.5 mole % of CFC-114a and 0.7 mole % of other unidentified compounds. The molar yield of CFC-114a compared to the total amount of CFC-114a and HCFC-124 was 26.8%.

### Example 6

### Photochlorination of HFC-152a

In this experiment, the distance of the lamp from the coiled tube was 0.5 inch (1.3 cm). Feed gases consisting of HFC-152a at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 30.5 mole % of HFC-152a, 67.7 mole % of HCFC-142b, 1.0 mole % of HCFC-142, 0.2 mole % of HCFC-132b and 0.6 mole % of other unidentified compounds.

### Example 7

### Photochlorination of HFC-152a

In this experiment, the distance of the lamp from the coiled tube was 3.0 inches (7.6 cm). Feed gases consisting of HFC-152a at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 27.9 mole % of HFC-152a, 70.1 mole % of HCFC-142b, 0.9 mole % of HCFC-142, 0.2 mole % of HCFC-132b and 0.9 mole % of other unidentified compounds.

### Example 8

### Photochlorination of HFC-134

Feed gases consisting of HFC-134 at a flow rate of 5.0 sccm (8.3(10)⁻⁸ m³/sec) and chlorine gas at a flow rate of 2.5 sccm (4.2(10)⁻⁸ m³/sec) were introduced into the PTFE tubing. After exposure to light for one hour, the product was analyzed and found to contain 43.4 mole % of HFC 134, 50.8 mole % of HCFC-124a, 5.3 mole % of CFC-114 and 0.5 mole % of other unidentified compounds. The molar yield of CFC-114 compared to the total amount of CFC-114 and HCFC-124a was 9.4%.

### Example 9

### Photochlorination of HFC 236ea

Feed gases consisting of HFC-236ea at a flow rate of 5.0 sccm (8.3(10)⁻⁸ m³/sec) and chlorine gas at a flow rate of 2.5 sccm (4.2(10)⁻⁸ m³/sec) were introduced into the PTFE tubing. After exposure to light for one hour, the product was analyzed and found to contain 59.7 mole % of HFC-236ea, 5.6 mole % of HCFC-226ba, 33.6 mole % of HCFC-226ea, 0.7 mole % of CFC-216ba and 0.4 mole % of other unidentified compounds.

### Example 10

### Photochlorination of HFC 236ea

Feed gases consisting of HFC-236ea at a flow rate of 5.0 sccm (8.3(10)⁻⁸ m³/sec) and chlorine gas at a flow rate of 7.5 sccm (1.3(10)⁻⁷ m³/sec) were introduced into the PTFE tubing. After exposure to light for one hour, the product was analyzed and found to contain 61.4 mole % of HFC-236ea, 5.5 mole % of HCFC-226ba, 31.9 mole % of HCFC-226ea, 0.7 mole % of CFC-216ba and 0.5 mole % of other unidentified compounds.

### Example 11

### Photochlorination of HFC-245fa

HFC-245fa was analyzed prior to chlorination to have a purity of 99.8%. Feed gases consisting of HFC-245fa at a flow rate of 3.5 sccm (5.8(10)⁻⁸ m³/sec) and chlorine gas at a flow rate of 3.5 sccm (5.8(10)⁻⁸ m³/sec) were introduced into the PTFE tubing. After exposure to light for one hour, the product was analyzed and found to contain 65.8 mole % of HFC-245fa, 33.0 mole % of HCFC-235fa, and 1.2 mole % of other unidentified compounds.

### Example 12

### Photochlorination of HFC-245fa

HFC-245fa was analyzed prior to chlorination to have a purity of 99.8%. Feed gases consisting of HFC-245fa at a flow rate of 5.0 sccm (8.3(10)⁻⁸ m³/sec) and chlorine gas at a flow rate of 2.5 sccm (4.2(10)⁻⁸ m³/sec) were introduced into the FEP tubing. After exposure to light for one hour, the product was analyzed and found to contain 18.6 mole % of HFC-245fa, 81.0 mole % of HCFC-235fa, and 0.4 mole % of other unidentified compounds.

What is claimed is:

1. A process for increasing the fluorine content of at least one compound selected from halohydrocarbons and hydrocarbons, comprising:
(a) directing light from a light source through the wall of a reactor to interact with reactants comprising chlorine and said at least one compound in said reactor, thereby producing a halogenated hydrocarbon having increased chlorine content by photochlorination; and
(b) reacting said halogenated hydrocarbon produced by the photochlorination in (a) with HF; wherein the light directed through the reactor wall is directed through a poly(perhaloolefin) polymer.

2. The process of claim 1 wherein the poly(perhaloolefin) polymer is a perfluorinated polymer.

3. The process of claim 2 wherein the poly(perhaloolefin) polymer is poly(tetrafluoroethylene).

4. The process of claim 2 wherein the poly(perhaloolefin) polymer is a copolymer of tetrafluoroethylene and hexafluoropropylene.

5. The process of claim 1 wherein in (a) CF₃CH₂CHF₂ is photochlorinated to CF₃CH₂CClF₂; and in (b) CF₃CH₂CClF₂ is reacted with HF to produce CF₃CH₂CF₃.

6. The process of claim 1 wherein in (a) CF₃CH₂F is photochlorinated to CF₃CHClF; and in (b) CF₃CHClF is reacted with HF to produce CF₃CHF₂.

7. The process of claim 1 wherein in (a) CHF₂CHF₂ is photochlorinated to CHF₂CClF₂; and in (b) CHF₂CClF₂ is reacted with HF to produce CF₃CHF₂.
